(19) United States
(12) Patent Application Publication
(10) Pub. No.: US 2025/0099535 A1
(43) Pub. Date: Mar. 27, 2025

(54) GEPOTIDACIN AND VANCOMYCIN FOR USE IN THE TREATMENT OF AN INFECTION CAUSED BY STAPHYLOCOCCUS SAPROPHYTICUS

(71) Applicant: GlaxoSmithKline Intellectual Property Development Limited, Stevenage (GB)

(72) Inventors: Joshua WEST, Collegeville, PA (US); Nicole SCANGARELLA-OMAN, Collegeville, PA (US)

(21) Appl. No.: 18/728,991

(22) PCT Filed: Jan. 19, 2023
(86) PCT No.: PCT/EP2023/051177

§ 371 Date: Jul. 15, 2024

Related U.S. Application Data
(60) Provisional application No. 63/301,522, filed on Jan. 21, 2022.

(51) Int. Cl. A61K 38/14; A61K 31/4985; A61P 31/04

(52) U.S. Cl. CPC A61K 38/14; A61K 31/4985; A61P 31/04

(57) ABSTRACT

The present invention relates to methods for treating bacterial infections by Staphylococcus saprophyticus, which comprises administration of gepotidacin or pharmaceutically acceptable salts thereof, and vancomycin or a pharmaceutically acceptable salt thereof to a human in need thereof. Novel pharmaceutical combinations, compositions, resistance guided therapies and/or corresponding uses thereof are also disclosed.

### STATEMENT REGARDING FEDERALLY SPONSORED RESEARCH

This invention was made with Government support under The United States Of America Department Of Health And Human Services Assistant Secretary For Preparedness And Response, Biomedical Advanced Research and Development Authority (BARDA), within the Office of the Assistant Secretary for Preparedness and Response in the U.S. Department of Health and Human Services Agreement No.: HHSO100201300011C. The government has certain rights in this invention.

### FIELD OF THE INVENTION

The present invention relates to methods of treatment, pharmaceutical combinations or compositions, combination or resistance guided therapies and/or corresponding uses thereof for treating a bacterial infection caused by Staphylococcus saprophyticus, which comprises administration of gepotidacin or pharmaceutically acceptable salts thereof and vancomycin or pharmaceutically acceptable salts thereof.

### BACKGROUND TO THE INVENTION

The misuse and overuse of antibiotics has led to concerning levels of global resistance rates across diseases, resulting in a world-wide call to action. Infections caused by multidrug-resistant organism(s) represent a major public health burden, not just in terms of morbidity and mortality, but also in terms of increased expenditure on patient management and implementation of infection control measures. The problem of antibacterial resistance is compounded by the existence of bacterial strains resistant to multiple antibacterials.

Antibiotics are routinely used for the treatment of urinary tract infections (UTIs), which very common, with approximately 11% of women above the age of 18 years of age experiencing at least 1 episode per year. Of these, half will experience more than 1 recurrent episode over their lifetime. UTI is caused by a variety of uropathogens including Staphylococcus saprophyticus which is estimated to be the causative agent in 5% to 15% of community-acquired UTIs.

Vancomycin is a glycopeptide antibiotic which is active against aerobic and anaerobic Gram-positive bacteria. Vancomycin is often used for patients with UTIs caused by Gram-positive bacterial infection. However, recent years have seen increasing bacterial resistance to vancomycin, particularly by enterococci, which presents a serious a medical and public health risk.

To date, a variety of antibacterial drugs have been developed which have become clinically extremely important antimicrobial drugs. Researchers at GlaxoSmithKline described a novel class of antibacterial agents that target type IIA topoisomerases [see Nature, Volume 466, pages 935-940 (19 Aug. 2010) and Gibson et al. Mechanistic and Structural Basis for the Actions of the Antibacterial Gepotidacin against Staphylococcus aureus Gyrase, ACS Infectious Disease, 2019, 5, 570-581] that has shown activity against a broad spectrum of gram-positive and gram-negative bacteria. International Patent Publication WO 2008/128942 and U.S. Pat. No. 8,389,524, hereby incorporated by reference in their entirety, disclose tricyclic nitrogen containing compounds as antibacterial compounds, pharmaceutical compositions and corresponding uses thereof.

There is a demand for development of novel pharmaceutical combinations or compositions, resistance guided therapies and/or uses thereof containing a combination of antibiotic or antimicrobial agents with different mechanisms of action for treating bacterial infections, which demonstrate synergistic and bactericidal effects leading to less development of resistance. However, any combination of agents should not have an antagonistic interaction.

The present invention is directed to overcoming these and other problems encountered in the art.

### SUMMARY OF THE INVENTION

Gepotidacin has now been unexpectedly found to be synergistically effective against Staphylococcus saprophyticus when acting together with the antibiotic vancomycin, with no negative interaction.

Thus, the present invention provides a method for treating an infection caused by Staphylococcus saprophyticus in a human in need thereof, comprising administering to said human a therapeutically effective amount of gepotidacin or a pharmaceutically acceptable salt thereof, with a therapeutically effective amount of vancomycin or a pharmaceutically acceptable salt thereof.

The present invention also provides vancomycin or a pharmaceutically acceptable salt thereof for use in the treatment of an infection in a human caused by Staphylococcus saprophyticus by co-administration with gepotidacin or a pharmaceutically acceptable salt thereof.

The present invention also provides gepotidacin or a pharmaceutically acceptable salt thereof for use in the treatment of an infection in a human caused by Staphylococcus saprophyticus by co-administration with vancomycin or a pharmaceutically acceptable salt thereof.

The present invention also provides use of gepotidacin or a pharmaceutically acceptable salt thereof, and vancomycin or a pharmaceutically acceptable salt thereof, in the manufacture of a medicament for the treatment of an infection in a human caused by Staphylococcus saprophyticus.

The present invention also provides use of vancomycin or a pharmaceutically acceptable salt thereof in the manufacture of a medicament for the treatment of an infection in a human caused by Staphylococcus saprophyticus, by co-administration with gepotidacin or a pharmaceutically acceptable salt thereof.

The present invention also provides use of gepotidacin or a pharmaceutically acceptable salt thereof in the manufacture of a medicament for the treatment of an infection in a human caused by Staphylococcus saprophyticus, by co-administration with vancomycin or a pharmaceutically acceptable salt thereof.

The present invention also provides a kit comprising gepotidacin or a pharmaceutically acceptable salt thereof, and vancomycin or a pharmaceutically acceptable salt thereof, for use in the treatment of an infection in a human caused by Staphylococcus saprophyticus.

The present invention also provides a pharmaceutical combination which comprises gepotidacin or a pharmaceutically acceptable salt thereof and vancomycin or a pharmaceutically acceptable salt thereof.

The present invention also provides a pharmaceutical composition which comprises gepotidacin or a pharmaceutically acceptable salt thereof, vancomycin or a pharmaceutically acceptable salt thereof, and at least one pharmaceutically acceptable excipient(s).

### DESCRIPTION OF DRAWINGS/FIGURES

FIGS. 1-5 show the bactericidal time-kill curves for each of the 5 isolates of Staphylococcus saprophyticus tested in the Example herein. In each of the Figures, A shows the kill curves at ¼× MIC gepotidacin and ½× MIC vancomycin; B shows the kill curves at 1× MIC gepotidacin and 1× MIC vancomycin.

FIG. 1 shows the bactericidal time-kill curves for Staphylococcus saprophyticus 1106006 (WT). FIG. 1A shows the the kill curves at ¼× MIC gepotidacin and ½× MIC vancomycin. FIG. 1B shows the kill curves at 1× MIC gepotidacin and 1× MIC vancomycin.

FIG. 2 shows the bactericidal time-kill curves for Staphylococcus saprophyticus 1113726 (WT). FIG. 2A shows the the kill curves at ¼× MIC gepotidacin and ½× MIC vancomycin. FIG. 2B shows the kill curves at 1× MIC gepotidacin and 1× MIC vancomycin.

FIG. 3 shows the bactericidal time-kill curves for Staphylococcus saprophyticus 1115244 (WT). FIG. 3A shows the the kill curves at ¼× MIC gepotidacin and ½× MIC vancomycin. FIG. 3B shows the kill curves at 1× MIC gepotidacin and 1× MIC vancomycin.

FIG. 4 shows the bactericidal time-kill curves for Staphylococcus saprophyticus 1125669 (WT). FIG. 4A shows the the kill curves at ¼× MIC gepotidacin and ½× MIC vancomycin. FIG. 4B shows the kill curves at 1× MIC gepotidacin and 1× MIC vancomycin.

FIG. 5 shows the bactericidal time-kill curves for Staphylococcus saprophyticus 1129086 (WT). FIG. 5A shows the the kill curves at ¼× MIC gepotidacin and ½× MIC vancomycin. FIG. 5B shows the kill curves at 1× MIC gepotidacin and 1× MIC vancomycin.

### DETAILED DESCRIPTION OF THE INVENTION

The terms “antimicrobial”, “antibiotic” and “antibacterial” refer to any natural or synthetic compound which kills or inhibits the growth of a microorganism.

Antibiotic resistance occurs when bacteria change in response to the use of antibiotics, making them ineffective.

As would be understood by the skilled person, the term “vancomycin” as used herein encompasses all forms of vancomycin including vancomycin hydrochloride.

Gepotidacin is a first-in-class, novel triazaacenaphthylene antibiotic with the ability to selectively inhibit bacterial DNA replication by a means not utilized by any currently approved human therapeutic agent, therefore providing the opportunity to address an unmet medical need. Gepotidacin and its racemic form is disclosed in WO 2008/128942 (herein incorporated in its entirety). Gepotidacin is (2R)-2-({4-[(3,4-dihydro-2H-pyrano[2,3-c]pyridin-6-ylmethyl) amino]-1-piperidinyl}methyl)-1,2-dihydro-3H,8H-2a,5,8a-triazaacenaphthylene-3,8-dione:

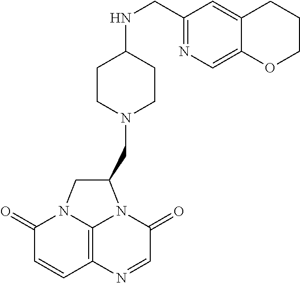 [SMILES: O=c1ccc2ncc(=O)n3c2n1C[C@H]3CN1CCC(NCc2cc3c(cn2)OCCC3)CC1]

The methods and combinations of the present invention are based on the combination of gepotidacin or a pharmaceutically acceptable salt thereof, and vancomycin or a pharmaceutically acceptable salt thereof. Combination therapy is a treatment in which a patient is given two or more drugs (or other therapeutic agents) for a single disease or when multiple diseases or pathogens are suspected or known to be present. Combination antibiotic therapy is used in patients due to widespread emergence of multidrug resistant (MDR) organisms. Multidrug resistance may be defined as lack of susceptibility to at least one agent in three or more antibiotic categories. Antimicrobials or antibacterials are frequently used in combination, so inhibitory drug interactions between the agents are undesirable.

Combination therapy may have the advantages of broadening antibacterial spectrum, providing synergistic effects, and discouraging the emergence of resistance.

The studies in the present application show a synergistic effect when combining gepotidacin and vancomycin against Staphylococcus saprophyticus.

Thus, in the first aspect, the present invention provides a method for treating an infection caused by Staphylococcus saprophyticus in a human in need thereof, comprising administering to said human a therapeutically effective amount of gepotidacin or a pharmaceutically acceptable salt thereof, with a therapeutically effective amount of vancomycin or a pharmaceutically acceptable salt thereof.

As used herein, “therapeutically effective amount” means a nontoxic but sufficient amount of the active ingredient to provide the desired effect.

As used herein, “caused by Staphylococcus saprophyticus” may mean that Staphylococcus saprophyticus has been identified as being the cause of an infection, or part of the cause of an infection (i.e. associated with the infection); or it may mean that Staphylococcus saprophyticus is suspected or strongly suspected to be the cause of the infection, or part of the cause of the infection, due to identification of symptoms and other factors such as patient history or local epidemiology.

In any of the aspects of the present invention, in one embodiment, the infection is urinary tract infection (UTI). Urinary tract infection is an infection of the bladder (also known as “cystitis”). In one embodiment, the infection is uncomplicated UTI (uUTI), which is defined as “acute, sporadic or recurrent lower (uncomplicated cystitis) and/or upper (uncomplicated pyelonephritis) UTI, limited to non-pregnant women with no known relevant anatomical and functional abnormalities within the urinary tract or comorbidities” in the “Guidelines on Urological Infections” (European Urological Society), https://uroweb.org/guideline/urological-infections. Symptoms of uUTI can come on suddenly, and can include: frequent and strong urge to urinate even after emptying the bladder; dysuria, a painful or burning sensation when urinating; foul- or strong-smelling urine; cloudy urine; a sensation of pressure, bladder fullness, or cramping in the middle of the lower abdomen or back; a low-grade fever; chills; and/or the presence of blood in the urine. uUTI is generally seen in otherwise healthy subjects, mostly female, without relevant structural and functional abnormalities within the urinary tract, kidney diseases or comorbidity that could lead to more serious outcomes and require additional attention.

In one embodiment, the present invention provides a method for treating UTI in a human in need thereof, comprising administering to said human a therapeutically effective amount of gepotidacin or a pharmaceutically acceptable salt thereof, with a therapeutically effective amount of vancomycin or a pharmaceutically acceptable salt thereof.

In one embodiment, the present invention provides a method for treating UTI caused by Staphylococcus saprophyticus in a human in need thereof, comprising administering to said human a therapeutically effective amount of gepotidacin or a pharmaceutically acceptable salt thereof, with a therapeutically effective amount of vancomycin or a pharmaceutically acceptable salt thereof.

In any aspect of the present invention, the gepotidacin or a pharmaceutically acceptable salt thereof, and vancomycin or a pharmaceutically acceptable salt thereof, may be administered sequentially (i.e. serial administration), concurrently (i.e. co-administration) or simultaneously (i.e. simultaneous administration) in separate or combined pharmaceutical formulations by any convenient route.

In one embodiment, the gepotidacin or a pharmaceutically acceptable salt thereof, and vancomycin or a pharmaceutically acceptable salt thereof, are administered sequentially.

The administration of vancomycin may follow recommended treatment guidelines.

Thus for example, vancomycin or its pharmaceutically acceptable salt (such as vancomycin HCl) may be given orally 125 mg every 6 hours for 10 days; or 500 mg every 6 hours for 10 days.

Given the synergistic effect of vancomycin with gepotidacin as shown in the present application, the length of period of vancomycin administration may be reduced from 10 days to, for example, 1, 2, 3, 4, 5, 6, 7, 8 or 9 days.

In one embodiment, for any aspect of the present invention, the human is administered gepotidacin or a pharmaceutically acceptable salt thereof for 1, 2, 3, 4, 5, 6, 7, 8, 9 or 10 days. In one embodiment, for any aspect of the present invention, the gepotidacin or a pharmaceutically acceptable salt thereof is administered at 1500 mg (measured as free base), b.i.d. (total daily dose 3000 mg measured as free base) for 5 days. In another embodiment, for any aspect of the present invention, the gepotidacin or a pharmaceutically acceptable salt thereof is administered at 3000 mg once. In another embodiment, for any aspect of the present invention, the gepotidacin or a pharmaceutically acceptable salt thereof is administered at two doses of 3000 mg each (measured as free base), 6-12 or 10-12 hours apart.

In another aspect, the present invention provides a method for the treatment of an infection caused by Staphylococcus saprophyticus in a human in need thereof, comprising administering to said human a therapeutically effective amount of vancomycin or a pharmaceutically acceptable salt thereof followed by a therapeutically effective amount of gepotidacin or a pharmaceutically acceptable salt thereof.

In one embodiment, the human is administered vancomycin or a pharmaceutically acceptable salt thereof for 1, 2, 3, 4, 5, 6, 7, 8, 9, 10, 11, 12, 13 or 14 days, followed by gepotidacin or a pharmaceutically acceptable salt thereof for 1, 2, 3, 4, 5, 6, 7, 8, 9, 10, 11, 12, 13 or 14 days. In one embodiment, the human is administered vancomycin or a pharmaceutically acceptable salt thereof for 7, 8, 9, 10, 11, 12, 13 or 14 days, followed by gepotidacin or a pharmaceutically acceptable salt thereof for 7, 8, 9, 10, 11, 12, 13 or 14 days.

In another aspect, the present invention provides a method for the treatment of an infection caused by Staphylococcus saprophyticus in a human in need thereof, comprising administering to said human a therapeutically effective amount of gepotidacin or a pharmaceutically acceptable salt thereof followed by a therapeutically effective amount of vancomycin or a pharmaceutically acceptable salt thereof.

In one embodiment, the human is administered gepotidacin or a pharmaceutically acceptable salt thereof for 1, 2, 3, 4, 5, 6, 7, 8, 9, 10, 11, 12, 13 or 14 days, followed by vancomycin or a pharmaceutically acceptable salt thereof for 1, 2, 3, 4, 5, 6, 7, 8, 9, 10, 11, 12, 13 or 14 days. In one embodiment, the human is administered gepotidacin or a pharmaceutically acceptable salt thereof for 7, 8, 9, 10, 11, 12, 13 or 14 days, followed by vancomycin or a pharmaceutically acceptable salt thereof for 7, 8, 9, 10, 11, 12, 13 or 14 days.

In one embodiment, for any aspect of the present invention, the total length of treatment is equal to or fewer than 1, 2, 3, 4, 5, 6, 7, 8, 9 or 10 days.

In another aspect, the present invention provides a method of treating an infection caused by Staphylococcus saprophyticus in a human in need thereof, comprising administering to said human a pharmaceutical composition comprising (a) a therapeutically effective amount of gepotidacin or a pharmaceutically acceptable salt thereof and (b) a therapeutically effective amount of vancomycin or a pharmaceutically acceptable salt thereof.

In one aspect, the present invention relates to a combination therapy for treating a bacterial infection caused by Staphylococcus saprophyticus, comprising administering a therapeutically effective amount of gepotidacin or a pharmaceutically acceptable salt thereof, in combination with a therapeutically effective amount of vancomycin or a pharmaceutically acceptable salt thereof, to a human in need thereof.

In one embodiment, the infection is UTI. In another embodiment, the infection is uUTI.

In one embodiment, the present invention provides a combination therapy for treating UTI, comprising administering a therapeutically effective amount of gepotidacin or a pharmaceutically acceptable salt thereof, in combination with a therapeutically effective amount of vancomycin or a pharmaceutically acceptable salt thereof, to a human in need thereof.

In one embodiment, the present invention provides a combination therapy for treating UTI, comprising administering a therapeutically effective amount of gepotidacin or a pharmaceutically acceptable salt thereof, in combination with a therapeutically effective amount of vancomycin or a pharmaceutically acceptable salt thereof, to a human in need thereof.

In one embodiment, the present invention provides a combination therapy for treating UTI caused by Staphylococcus saprophyticus, comprising administering a therapeutically effective amount of gepotidacin or a pharmaceutically acceptable salt thereof, in combination with a therapeutically effective amount of vancomycin or a pharmaceutically acceptable salt thereof, to a human in need thereof.

In one embodiment, the present invention provides a combination therapy for treating UTI caused by Staphylococcus saprophyticus, comprising administering a therapeutically effective amount of gepotidacin or a pharmaceutically acceptable salt thereof, in combination with a therapeutically effective amount of vancomycin or a pharmaceutically acceptable salt thereof, to a human in need thereof.

The gepotidacin or a pharmaceutically acceptable salt thereof may be present in a pharmaceutical composition which comprises gepotidacin or a pharmaceutically acceptable salt thereof and at least one pharmaceutically acceptable excipient(s). Similarly, the vancomycin or a pharmaceutically acceptable salt thereof may be present in a corresponding pharmaceutical composition of vancomycin or a pharmaceutically acceptable salt thereof which comprises vancomycin or a pharmaceutically acceptable salt thereof and at least one pharmaceutically acceptable excipient(s).

In another aspect, the present invention relates to a combination or resistance guided therapy for treating a bacterial infection caused by Staphylococcus saprophyticus, comprising administering a therapeutically effective amount of gepotidacin or a pharmaceutically acceptable salt thereof, in combination with a therapeutically effective amount of vancomycin or a pharmaceutically acceptable salt thereof, to a human in need thereof. The gepotidacin or a pharmaceutically acceptable salt thereof may be present in a pharmaceutical composition which comprises gepotidacin or a pharmaceutically acceptable salt thereof and at least one pharmaceutically acceptable excipient(s). Similarly, the vancomycin or a pharmaceutically acceptable salt thereof may be present in a corresponding pharmaceutical composition of vancomycin or a pharmaceutically acceptable salt thereof which comprises vancomycin or a pharmaceutically acceptable salt thereof and at least one pharmaceutically acceptable excipient(s). In one embodiment, the infection is UTI. In one embodiment, the infection is uUTI.

As would be understood by the skilled person, as used herein, “resistance guided therapy” means a course of therapy, the direction of which is guided by knowledge of the phenotypic or genotypic susceptibility of the microorganism to a given antibiotic, for example as described in Bradshaw et al, The Journal of Infectious Diseases, Volume 216, Issue suppl_2, 15 Jul. 2017, Pages S412-S419. Detecting Staphylococcus saprophyticus in an infection, then detecting the resistance of the Staphylococcus saprophyticus strain to certain antibiotics, in advance or during the course of treatment, has the advantage of potentially reducing the patient's exposure to ineffective antibiotics that may lead to resistance. Identification of Staphylococcus saprophyticus may be performed by any suitable genotypic or phenotypic means, such as by NAAT.

Thus in one aspect, the present invention provides a resistance guided therapy for treating a bacterial infection caused by Staphylococcus saprophyticus, comprising administering a therapeutically effective amount of gepotidacin or a pharmaceutically acceptable salt thereof, in combination with a therapeutically effective amount of vancomycin or a pharmaceutically acceptable salt thereof, to a human in need thereof.

The combination or resistance guided therapy of the present invention may be achieved by simultaneous administration, co-administration or serial administration of the two components.

In one aspect, the present invention relates to a combination or resistance guided therapy for treating a bacterial infection caused by Staphylococcus saprophyticus, where the bacterial infection is UTI.

In one aspect, the present invention relates to a combination or resistance guided therapy for treating a bacterial infection caused by Staphylococcus saprophyticus, where UTI is uUTI.

In one aspect, the present invention relates to a combination or resistance guided therapy for treating a bacterial infection caused by Staphylococcus saprophyticus, where each of the components are administered orally.

In one aspect, the present invention relates to a combination or resistance guided therapy for treating a bacterial infection caused by Staphylococcus saprophyticus, which comprises simultaneous administration, co-administration or serial administration of a therapeutically effective amount of gepotidacin or a pharmaceutically acceptable salt thereof and vancomycin or a pharmaceutically acceptable salt thereof, to a human in need thereof; where use of vancomycin or a pharmaceutically acceptable salt thereof results in a synergistic effect; and/or also aids in protecting against development of resistance to either gepotidacin or a pharmaceutically acceptable salt thereof or vancomycin or a pharmaceutically acceptable salt thereof without interfering in its respective activity.

In one aspect, the present invention relates to a combination or resistance guided therapy for treating a bacterial infection caused by Staphylococcus saprophyticus, which comprises simultaneous administration, co-administration or serial administration of a therapeutically effective amount of a pharmaceutical composition which comprises gepotidacin or a pharmaceutically acceptable salt thereof and at least one or more pharmaceutically acceptable excipient(s); and vancomycin or a pharmaceutically acceptable salt thereof, to a human in need thereof; where use of vancomycin or a pharmaceutically acceptable salt thereof results in a synergistic effect; and/or also aids in protecting against development of resistance to either gepotidacin or a pharmaceutically acceptable salt thereof or vancomycin or a pharmaceutically acceptable salt thereof without interfering in its respective activity.

In one aspect, the present invention relates to a combination or resistance guided therapy for treating UTI, which comprises administration of therapeutically effective amount of gepotidacin; and vancomycin or a pharmaceutically acceptable salt thereof to a human in need thereof; where use of vancomycin or a pharmaceutically acceptable salt thereof results in a synergistic effect; and/or also aids in protecting against development of resistance to either gepotidacin or a pharmaceutically acceptable salt thereof or vancomycin or a pharmaceutically acceptable salt thereof without interfering in their respective activities. In one embodiment, the UTI is uUTI.

In one aspect, the present invention relates to a combination or resistance guided therapy for treating UTI caused by Staphylococcus saprophyticus, which comprises administration of therapeutically effective amount of gepotidacin; and vancomycin or a pharmaceutically acceptable salt thereof to a human in need thereof; where use of vancomycin or a pharmaceutically acceptable salt thereof results in a synergistic effect; and/or also aids in protecting against development of resistance to either gepotidacin or a pharmaceutically acceptable salt thereof or vancomycin or a pharmaceutically acceptable salt thereof without interfering in their respective activities. In one embodiment, the UTI is uUTI.

In one aspect, the present invention relates to a resistance guided therapy for treating a bacterial infection caused by Staphylococcus saprophyticus, which comprises administration of a therapeutically effective amount of gepotidacin or a pharmaceutically acceptable salt thereof to a human in need thereof; and vancomycin or a pharmaceutically acceptable salt thereof, to a human in need thereof; where use of vancomycin or a pharmaceutically acceptable salt thereof results in a synergistic effect; and/or also aids in protecting against development of resistance to either gepotidacin or a pharmaceutically acceptable salt thereof or vancomycin or a pharmaceutically acceptable salt thereof without interfering in their respective activity. In any of the above aspects and the embodiments of the present invention, in one embodiment, the human is male. In one embodiment, the human is female.

In another aspect, the present invention provides vancomycin or a pharmaceutically acceptable salt thereof for use in the treatment of an infection caused by Staphylococcus saprophyticus by co-administration with gepotidacin or a pharmaceutically acceptable salt thereof.

In another aspect, the present invention provides gepotidacin or a pharmaceutically acceptable salt thereof for use in the treatment of an infection caused by Staphylococcus saprophyticus by co-administration with vancomycin or a pharmaceutically acceptable salt thereof.

In another aspect, the present invention provides use of gepotidacin or a pharmaceutically acceptable salt thereof, and vancomycin or a pharmaceutically acceptable salt thereof, in the manufacture of a medicament for the treatment of an infection caused by Staphylococcus saprophyticus.

Thus in one embodiment, the present invention provides vancomycin or a pharmaceutically acceptable salt thereof for use in the treatment of UTI caused by Staphylococcus saprophyticus by co-administration with gepotidacin or a pharmaceutically acceptable salt thereof.

In another aspect, the present invention provides gepotidacin or a pharmaceutically acceptable salt thereof for use in the treatment of UTI caused by Staphylococcus saprophyticus by co-administration with vancomycin or a pharmaceutically acceptable salt thereof.

In another aspect, the present invention provides use of vancomycin or a pharmaceutically acceptable salt thereof in the manufacture of a medicament for the treatment of an infection caused by Staphylococcus saprophyticus, by co-administration with gepotidacin or a pharmaceutically acceptable salt thereof.

In another aspect, the present invention provides use of gepotidacin or a pharmaceutically acceptable salt thereof in the manufacture of a medicament for the treatment of an infection caused by Staphylococcus saprophyticus, by co-administration with vancomycin or a pharmaceutically acceptable salt thereof.

The gepotidacin or a pharmaceutically acceptable salt thereof, and vancomycin or a pharmaceutically acceptable salt thereof, may be administered sequentially or simultaneously in separate or combined pharmaceutical formulations by any convenient route.

In another aspect, the present invention provides a kit comprising gepotidacin or a pharmaceutically acceptable salt thereof, and vancomycin or a pharmaceutically acceptable salt thereof, for use in the treatment of an infection caused by Staphylococcus saprophyticus.

In another aspect, the present invention provides a pharmaceutical combination, which comprises gepotidacin or a pharmaceutically acceptable salt thereof; and vancomycin or a pharmaceutically acceptable salt thereof.

In another aspect, the present invention provides a pharmaceutical composition, which comprises gepotidacin or a pharmaceutically acceptable salt thereof, vancomycin or a pharmaceutically acceptable salt thereof, and at least one pharmaceutically acceptable excipient(s).

In another aspect, the present invention provides a pharmaceutical combination of gepotidacin or a pharmaceutically acceptable salt thereof, and vancomycin or a pharmaceutically acceptable salt thereof.

In another aspect, the present invention relates to a pharmaceutical combination, which comprises gepotidacin or a pharmaceutically acceptable salt thereof and vancomycin or a pharmaceutically acceptable salt thereof for use in combination or resistance guided therapy as described in the present invention.

In another aspect, the present invention relates to a pharmaceutical composition comprising gepotidacin or a pharmaceutically acceptable salt thereof, vancomycin or a pharmaceutically acceptable salt thereof, and at least one pharmaceutically acceptable excipient(s) for use in combination or resistance guided therapy as described in the present invention.

In another aspect, the present invention provides a combination of gepotidacin or a pharmaceutically acceptable salt thereof, and vancomycin or a pharmaceutically acceptable salt thereof, for use in the treatment of an infection caused by Staphylococcus saprophyticus.

In another aspect, the present invention provides a pharmaceutical composition comprising gepotidacin or a pharmaceutically acceptable salt thereof, and vancomycin or a pharmaceutically acceptable salt thereof, for use in the treatment of an infection caused by Staphylococcus saprophyticus.

In another aspect, the present invention relates to a use of a pharmaceutical combination or a pharmaceutical composition as defined in the present invention for the manufacture of a medicament for treating an infection caused by Staphylococcus saprophyticus, such as UTI.

In another aspect, the present invention relates to a use of a pharmaceutical combination according or a pharmaceutical composition as herein described for the manufacture of a medicament for treating UTI.

In another aspect, the present invention relates to a use of a pharmaceutical combination according or a pharmaceutical composition as herein described for the manufacture of a medicament for treating UTI caused by Staphylococcus saprophyticus.

In another aspect, the present invention relates to a use of a pharmaceutical combination or a pharmaceutical composition as herein described for the manufacture of a medicament for treating uUTI caused by Staphylococcus saprophyticus.

In another aspect, the present invention relates to a use of a pharmaceutical combination or a pharmaceutical composition as defined in the present invention for resistance guided therapy for treating a bacterial infection caused by Staphylococcus saprophyticus in a human in need thereof.

In another aspect, the present invention relates to a use of a pharmaceutical combination or a pharmaceutical composition as defined in the present invention for combination therapy for treating a bacterial infection caused by Staphylococcus saprophyticus in a human in need thereof.

In another aspect, the present invention relates to a use as defined in the present invention, wherein the bacterial infection is UTI, such as uUTI.

### Compounds Used in the Present Invention

WO2008/128942 discloses the preparation of the free base and the hydrochloride salt of gepotidacin.

It will be understood that the phrase “gepotidacin or a pharmaceutically acceptable salt thereof” is intended to encompass gepotidacin, a pharmaceutically acceptable salt of gepotidacin, a solvate of gepotidacin, or any pharmaceutically acceptable combination of these. Thus by way of non-limiting example used here for illustrative purpose, “gepotidacin or a pharmaceutically acceptable salt thereof” may include a pharmaceutically acceptable salt of gepotidacin that is further present as a solvate.

It will be understood that the phrase “vancomycin or a pharmaceutically acceptable salt thereof” is intended to encompass vancomycin, a pharmaceutically acceptable salt of vancomycin, a solvate of vancomycin, or any pharmaceutically acceptable combination of these. Thus by way of non-limiting example used here for illustrative purpose, “vancomycin or a pharmaceutically acceptable salt thereof” may include a pharmaceutically acceptable salt of vancomycin that is further present as a solvate.

As used herein, vancomycin or gepotidacin (or any pharmaceutically acceptable salt thereof of both) may be in any physical form thereof, including non-solid forms such as liquid or semi-solid forms, solid forms such as amorphous or crystalline forms, specific polymorphic forms and solvates including hydrates.

Suitable pharmaceutically acceptable salts include those described by Berge, Bighley and Monkhouse J.Pharm.Sci (1977) 66, pp 1-19.

For both gepotidacin and vancomycin, a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, y-hydroxybutyrates, glycollates, tartrates mandelates, and sulfonates, such as xylenesulfonates, methanesulfonates, propanesulfonates, naphthalene-1-sulfonates and naphthalene-2-sulfonates.

Pharmaceutically acceptable salts of gepotidacin include the acid addition salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. In one embodiment, in any aspect of the invention, the gepotidacin is gepotidacin free base or is gepotidacin methanesulphonate (mesylate).

Pharmaceutically acceptable salts of vancomycin include the acid addition salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. In one embodiment, in any aspect of the invention, the vancomycin is vancomycin hydrochloride.

The present invention includes within its scope all possible stoichiometric and non-stoichiometric salt forms.

### Pharmaceutical Compositions and Formulations

Pharmaceutical compositions and formulations acceptable and adaptable for use in methods and/or uses of the present invention are prepared using conventional art known pharmaceutical compositions, formulation or chemical materials, formulary excipients, preparation means, processes and/or methods and conventional techniques, etc.

In particular, gepotidacin or pharmaceutically acceptable salts, used in the present invention may be formulated for administration in any convenient way for use in human or veterinary medicine, by analogy with other antibacterials/antitubercular compounds.

The pharmaceutical compositions used in the present invention may be formulated for administration by any route and include those in a form adapted for oral, topical or parenteral use and may be used in mammals including humans.

The compositions may be in the form of tablets, capsules, powders, granules, lozenges, suppositories, creams or liquid preparations, such as oral or sterile parenteral solutions or suspensions.

In one embodiment, the gepotidacin or pharmaceutically acceptable salt thereof of the present invention is in a tablet or a capsule form. In one embodiment, it is in a tablet form. In one embodiment, the tablet is a 750 mg tablet.

The vancomycin or a pharmaceutically acceptable salt thereof may be administered in any suitable form, including oral capsule (such as 125 mg or 250 mg), oral tablet, delayed or extended release oral tablet, delayed or extended release oral capsule, oral suspension (i.e. dry powder for reconstitution with water) or injectable solution.

Tablets and capsules for oral administration in the present invention may be in unit dose presentation form, and may contain conventional excipients such as binding agents, fillers, tabletting lubricants, disintegrants, or wetting agents. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

Moreover, the quantity of the compound or pharmaceutical composition used in the present invention administered will vary depending on the patient and the mode of administration and can be any effective amount.

In accordance with any of the methods of administration of the present invention, the term a “therapeutically effective amount”, as used herein, generally includes within its meaning a non-toxic but sufficient amount of the particular drug to which it is referring to provide the desired therapeutic effect. The exact amount required will vary from subject to subject depending on factors such as the patient's general health, the patient's age, etc.

Treatment regimens for the administration of the compounds and/or pharmaceutical compositions used in the present invention can also be determined readily by those with ordinary skill in art. The quantity of the compound and/or pharmaceutical composition used in the present invention administered may vary over a wide range to provide in a unit dosage an effective amount based upon the body weight of the patient per day to achieve the desired effect.

The compositions may contain from 0.1% by weight, preferably from 10-60% by weight, of the active material, depending on the method of administration. Where the compositions comprise dosage units, each unit will preferably contain from 50-1000 mg of the active ingredient. Unless otherwise noted, the amount of the active ingredient (i.e., gepotidacin) refers to that of gepotidacin free base.

The dosage of the gepotidacin or a pharmaceutically acceptable salt as employed for adult human treatment in the present invention will preferably range from 100 or 6000 mg per day, or 100 to 3000 mg per day, for instance 1500 mg per day or 3000 mg per day depending on the route and frequency of administration. Such a dosage corresponds to about 1.5 to about 100 or 50 mg/kg per day. Suitably the dosage is from 5 to 30 mg/kg per day. In one embodiment, the dosage is 1500 mg twice a day (i.e. 3000 mg per day). In one embodiment, the dosage is 3000 mg twice a day (i.e. 6000 mg per day).

Conventional administration methods may be suitable for use in the present invention.

Depending upon the treatment being effected, the compounds, and/or or compositions of the present invention can be administered orally, intravascularly, intraperitoneally, subcutaneously, intramuscularly or topically. Preferably, the composition is adapted for oral administration. In any of the above aspects of the present invention, in one embodiment, the gepotidacin or a pharmaceutically acceptable salt thereof and the vancomycin or a pharmaceutically acceptable salt thereof are administered orally.

The synergistic effect may help to shorten the patient's period of exposure to the first drug (which may be vancomycin or gepotidacin) to a few days, rather than a lengthy period, before switching to the other drug, or adding the other drug to the treatment regimen. This dual therapy may help protect gepotidacin from selection and/or spread of resistance as well as increase the potency of both compounds.

It is to be understood that the invention is not limited to the aspects or embodiments illustrated hereinabove and the right is reserved to the illustrated aspects or embodiments and all modifications coming within the scope of the following claims.

The various references to journals, patents, and other publications which are cited herein comprise the state of the art and are incorporated herein by reference as though fully set forth.

The Examples set forth below are illustrative of the present invention and are not intended to limit, in any way, the scope of the present invention.

### EXAMPLE

The example herein describes synergy, indifference and antagonism interactions between gepotidacin and vancomycin determined using reference in vitro broth microdilution checkerboards. Recent clinical isolates of Enterococcus faecalis and Staphylococcus saprophyticus (5 isolates per species) were tested against checkerboards of gepotidacin in combination with vancomycin. Synergy was observed for gepotidacin and vancomycin against 4 out of the 5 Staphylococcus saprophyticus isolates. Time-kill assays were performed when the observation of synergy was prevalent (>50% of isolates) for a species/drug combination.

Only gepotidacin and vancomycin against S. saprophyticus met this criterion. This synergistic activity between gepotidacin and vancomycin against S. saprophyticus was confirmed by time-kill for all 5 isolates at 1×MIC concentrations of the two drugs.

### Methods

### Baseline Broth Microdilutions

MIC values were determined in triplicate by Clinical and Laboratory Standards Institute (CLSI) M07 (2018) reference broth microdilution (BMD) using cation adjusted Mueller Hinton broth (CAMHB) to determine a consensus median baseline MIC value for each isolate and compound. As required by CLSI M100 (2021), quality control (QC) strains Escherichia coli ATCC 25922, Pseudomonas aeruginosa ATCC 27853, Staphylococcus aureus 29213, and Enterococcus faecalis ATCC 29212 were tested concomitantly with the clinical isolates. (QC data not shown).

### Checkerboard Panels

Broth microdilution panels were prepared according to methods described in the Clinical Microbiology Procedure Handbook, 4th Edition, 2016, Chapter 5.16. To assess interactions, Cation-adjusted Mueller-Hinton broth (CAMHB) was used for testing gepotidacin alone and in combination with other antimicrobial agents.

### Time-Kill Methods

Follow-up time-kill kinetic studies were carried out for all compound combinations where synergy was observed (FICs ≤0.5) among 50% of isolates within a species via checkerboard assays. CAMHB was used for the follow-up time-kill kinetic studies. Each organism was tested in media containing either compound alone, or both, at ½×, ¼×, and 1× their respective MICs. Time-kill concentration tubes were sampled at time 0 hours (T0), T2, T4, T8, and T24. Samples were serially diluted ten-fold up to 8 times in tubes with 0.15 mL of a 0.85% saline solution. A volume of 0.1 mL was then plated onto tryptic-soy agar with 5% sheep blood from the original sample and subsequent dilutions. Plates were incubated for 24 hours at 35° C. before quantifying the viable cell count for each tube at the specified times (T0-T24).

### Data Analysis

Interpretations of the antimicrobial combination interactions for checkerboards follow those outlined in the 4th edition of Clinical Microbiology Procedures Handbook (2016). Antimicrobial interaction categorical characterization was defined as synergy in checkerboard assays when the fractional inhibitory concentration (FIC) index was ≤0.5, indifferent when the FIC index was >0.5 to ≤4.0, and antagonistic when the FIC index was >4.0. Interactions other than synergy or antagonism are referred to as indifferent or indeterminant.

When the observation of synergy was prevalent for a species/drug combination (>50% of isolates tested for that species), time-kill assays were performed. To confirm synergy via time-kill, recorded cell counts were entered in an Excel spreadsheet and plotted against time to demonstrate time-kill curve kinetics. As described in the CLSI M26-A manual, bactericidal activity was defined as a 3-log10 decrease in CFU/mL below the starting inoculum that was maintained for 24 hours. Synergy via time-kill assays was defined as a ≥2 log10 CFU/mL decrease between the combination and the most active agent alone at 24 hours. The number of surviving organisms in the presence of the combination was required to be ≥2 log10 CFU/mL below the starting inocula and at least one of the drugs should not affect the growth curve of the tested organism. Indifference and antagonism were defined at 24 hours as a +/−1 log10 to <2 log10 kill compared to the most active agent alone and >1 log10 growth compared with the less active single agent, respectively.

### RESULTS

Activity against Enterococcus faecalis

No instances of synergy or antagonism were observed for gepotidacin and vancomycin tested against Enterococcus faecalis isolates (Table 1).

Activity against Staphylococcus saprophyticus

Gepotidacin demonstrated synergy with vancomycin for 4 out of 5 isolates. The 4 isolates that displayed synergy in the checkerboard assay had ΣFICmin values ranging from ≤0.25 to 0.31 (Table 1).

TABLE 1
MICs (μg/mL) and Fractional Inhibitory Concentration
Indices (FICI) for gepotidacin and vancomycin against
E. faecalis and S. saprophyticus

| | GEP | VANC | ΣFICI | ΣFICI | |
|---|---|---|---|---|---|
| Isolate | MIC | MIC | min | max | Synergy |
| S. saprophyticus 1106006 | 0.25 | 0.5 | 0.25 | 0.25 | Yes |
| S. saprophyticus 1113726 | 0.12 | 1 | 0.31 | 0.62 | Yes |
| S. saprophyticus 1115244 | 0.12 | 1 | 0.31 | 0.31 | Yes |
| S. saprophyticus 1125669 | 0.25 | 1 | 0.62 | 1.25 | No |
| S. saprophyticus 1129086 | 0.25 | 0.5 | 0.25 | 0.25 | Yes |
| E. faecalis 1097863 | 1 | 1 | 1.03 | 2.25 | No |
| E. faecalis 1103597 | 0.5 | 1 | 1.06 | 2.25 | No |
| E. faecalis 1103850 | 0.5 | 1 | 0.75 | 1.25 | No |
| E. faecalis 1111210 | 1 | 1 | 1.03 | 2.25 | No |
| E. faecalis 1124756 | 0.5 | 1 | 0.75 | 1.25 | No |

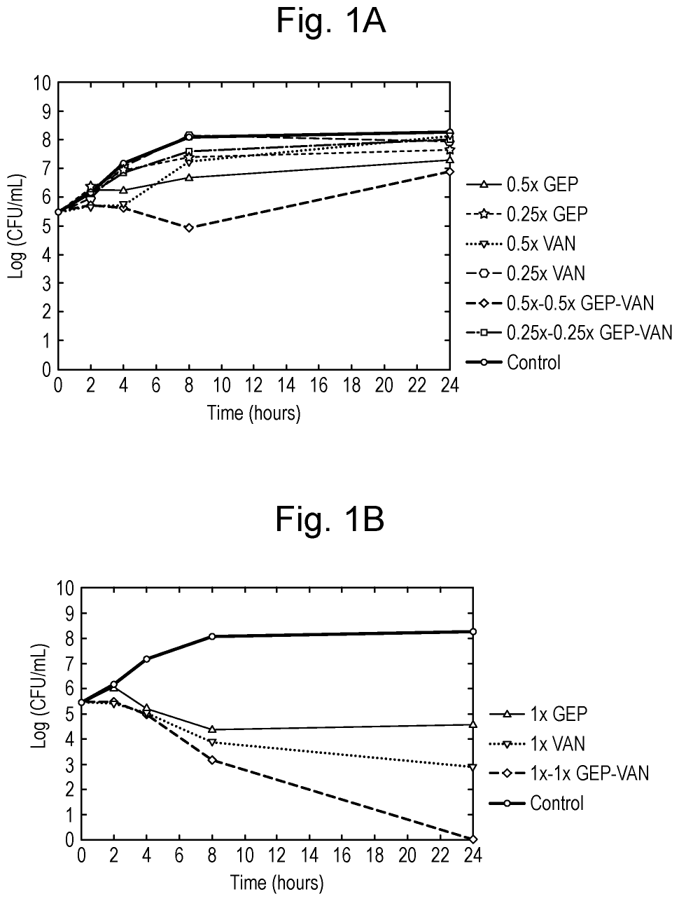

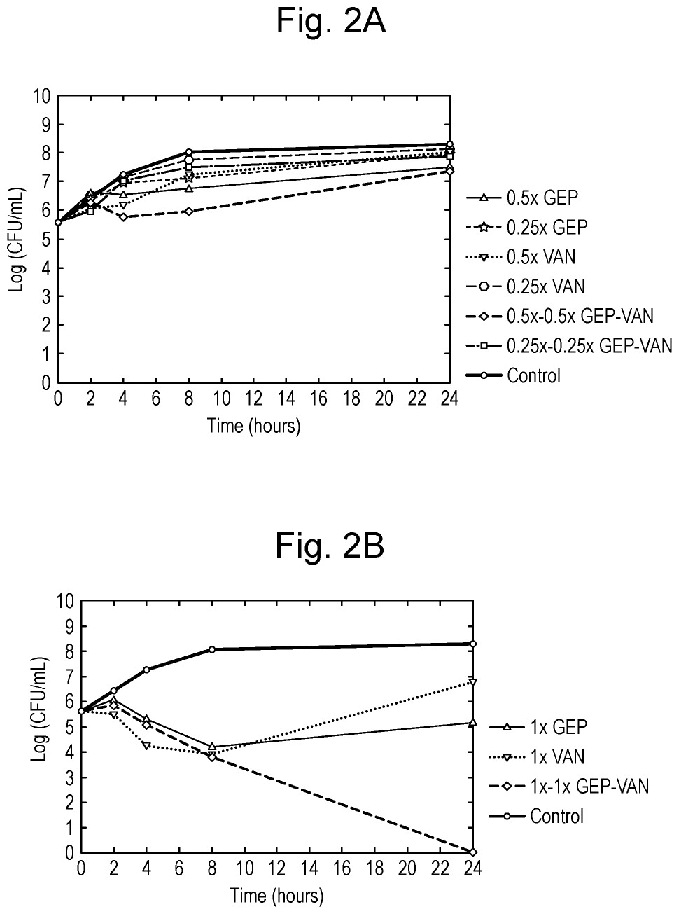

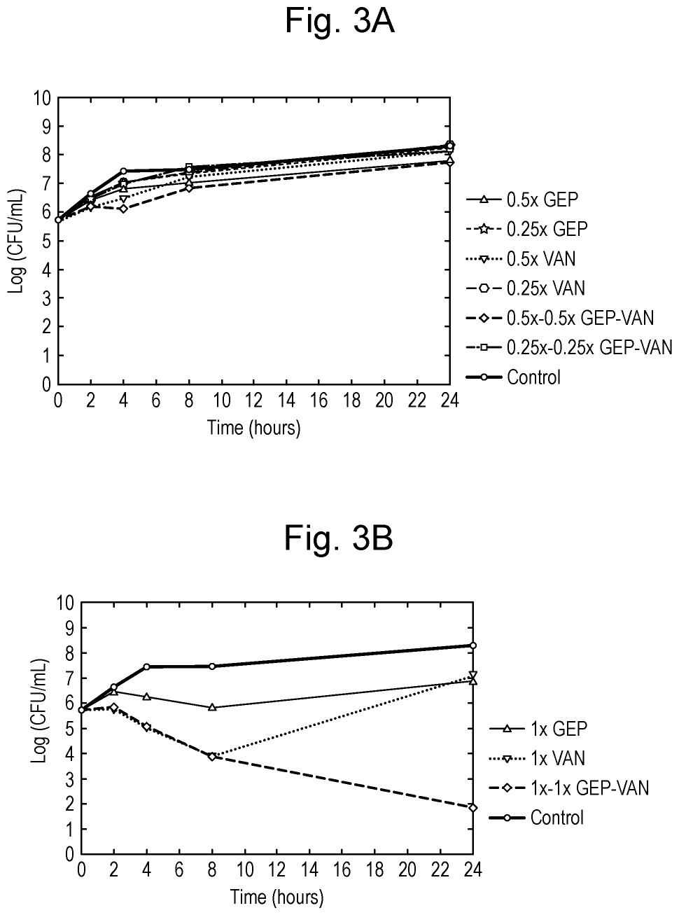

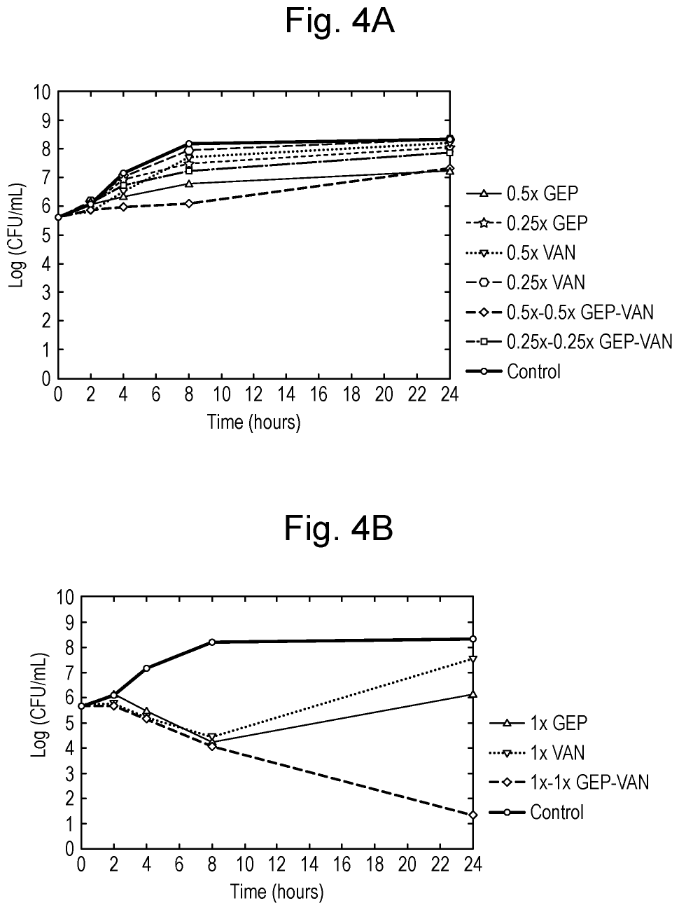

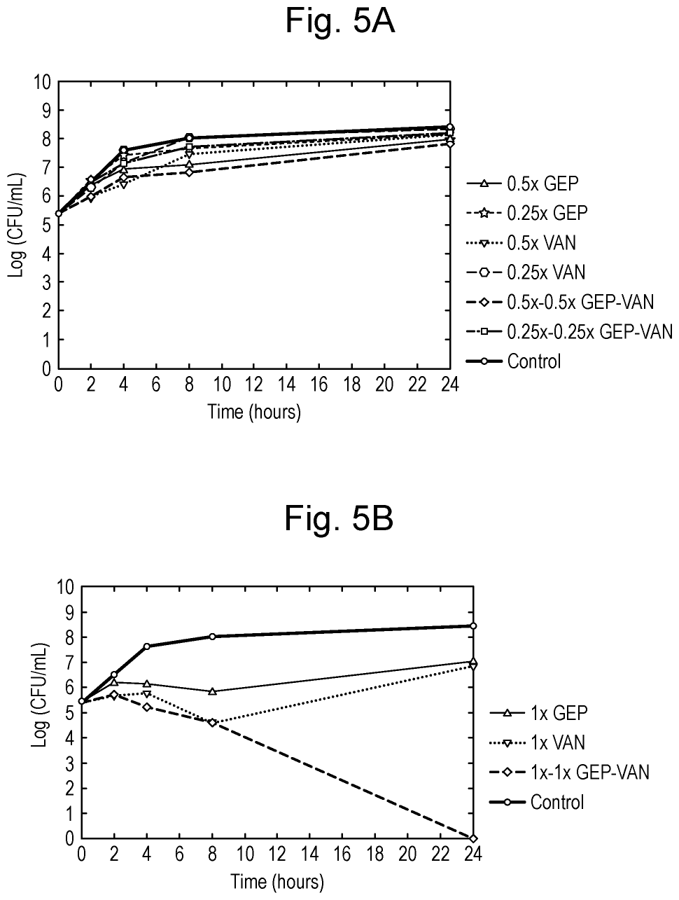

All 5 S. saprophyticus isolates were further evaluated via the time-kill assay. Synergy between gepotidacin and vancomycin, as defined as a ≥2 log10 CFU/mL decrease between the combination and the most active agent alone at 24 hours, was observed for all 5 isolates when both agents were tested at their respective 1× MIC concentrations (FIGS. 1-5). Time-kills under these conditions also met the definition of bactericidal activity (3-log10 decrease in CFU mL below the starting inoculum maintained for 24 hours), which was not achieved by either agent alone. The synergistic activity observed at the ¼× and ½× MIC conditions in the checkerboard assays was not confirmed in the time-kill studies.

No instances of antagonism were observed for combinations tested against Staphylococcus saprophyticus isolates.

1. A method for treating an infection caused by Staphylococcus saprophyticus in a human in need thereof, comprising administering to said human a therapeutically effective amount of gepotidacin or a pharmaceutically acceptable salt thereof, with a therapeutically effective amount of vancomycin or a pharmaceutically acceptable salt thereof.

2. A method as claimed in claim 1, wherein the infection is a urinary tract infection.

3. A method as claimed in claim 2, wherein the infection is uncomplicated urinary tract infection.

4. A method as claimed in claim 1, wherein the gepotidacin or a pharmaceutically acceptable salt thereof, and vancomycin or a pharmaceutically acceptable salt thereof, are administered sequentially.

5. A method as claimed in claim 1, wherein the gepotidacin is gepotidacin methanesulphonate.

6. A method as claimed in claim 1, wherein the vancomycin is vancomycin hydrochloride.

7. A method as claimed in claim 1, wherein the gepotidacin or a pharmaceutically acceptable salt thereof is administered orally.

8. A method as claimed in claim 1, wherein the vancomycin or a pharmaceutically acceptable salt thereof is administered orally.

9-14. (canceled)

15. A pharmaceutical combination which comprises gepotidacin or a pharmaceutically acceptable salt thereof and vancomycin or a pharmaceutically acceptable salt thereof.

16. A pharmaceutical composition which comprises gepotidacin or a pharmaceutically acceptable salt thereof, vancomycin or a pharmaceutically acceptable salt thereof, and at least one pharmaceutically acceptable excipient(s).

17-19. (canceled)
